(19) United States
(12) Patent Application Publication
(10) Pub. No.: US 2025/0237649 A1
(43) Pub. Date: Jul. 24, 2025

(54) COMPOSITIONS AND METHODS FOR HIP1-TARGETING INHIBITOR COMPOUNDS

(71) Applicant: Utah Valley University, Orem, UT (US)

(72) Inventors: Nathan E. Goldfarb, Orem, UT (US); Andrew D. Abell, Adelaide (AU); Nicholas C. Schumann, Adelaide (AU); Borja Lopez Perez, Santiago de Compostela (ES)

(21) Appl. No.: 19/177,139

(22) Filed: Apr. 11, 2025

Related U.S. Application Data
(63) Continuation-in-part of application No. 17/827,700, filed on May 28, 2022.
(60) Provisional application No. 63/217,172, filed on Jun. 30, 2021.

(51) Int. Cl. G01N 33/569; C07K 7/06; G01N 33/543; G01N 33/573

(52) U.S. Cl. CPC G01N 33/5695; C07K 7/06; G01N 33/54388; G01N 33/573; C07K 2319/60; G01N 2333/952

(57) ABSTRACT

Provided herein are inhibitor compounds targeting Hip1, with the inhibitor compounds comprising a tripeptide targeting sequence that directs the compound to the active site of Hip1 and a C-terminal electrophilic warhead conjugated to the targeting sequence, the warhead configured to inactive the enzyme. Further provided herein are embodiments of a treatment method and embodiments of an assay including embodiments of a rapid, point-of-care lateral flow assay (LFA).

### CROSS-REFERENCES TO RELATED APPLICATIONS

This application is a continuation-in-part of U.S. patent application Ser. No. 17/827,700, titled “COMPOSITION AND METHOD FOR HIP1-TARGETING INHIBITOR COMPOUNDS,” filed on May 28, 2022, which claims priority to and the benefit of United States Provisional Patent Application No. 63/217,172 titled “COMPOSITION AND METHOD FOR HIP1-TARGETING INHIBITOR COMPOUNDS,” filed on Jun. 30, 2021. Each of the foregoing documents is incorporated herein by reference in its entirety.

### FIELD OF THE INVENTION

Embodiments of the present disclosure relate to molecular targeting and more particularly relates to Hip1 inhibitor targeting drugs including for tuberculosis diagnosis and treatment.

### BACKGROUND

According to the World Health Organization, in 2018, 1.5 million people died from Mycobacterium tuberculosis (Mtb) (including 251,000 people with HIV). Active Mtb is highly infectious and requires fast and accurate diagnosis to provide effective treatment and control of proliferation. The current gold standard for a rapid diagnostic assay requires a result that can be identified quickly without the need of intensive lab work, and intuitive enough that it can be used by anyone, thereby meeting the ASSURED criterion (affordable, sensitive, specific, user-friendly, rapid, equipment-free, delivered). The diagnostic test QuantiFERON TB Gold Plus is the most sensitive to both active TB and latent TB but is limited due to the high cost and most importantly, the time required to obtain accurate results.

New drugs for the treatment of Tuberculosis (TB) are direly needed. This is due to the evolution of drug-resistant strains of Mtb, the causative agent of TB. Also, many of the current FDA-approved drugs used to treat drug resistant strains of TB are injectables, have toxic side effects, and require a six-month treatment regimen. Therefore, a need exists for a therapeutic compound narrowly targeting Mtb for an effective and rapid course of treatment. Beneficially, such a compound would be free of toxic side effects.

### SUMMARY

The foregoing illustrates an ongoing need for an effective, short-term treatment for Mtb that can also minimize or be free of toxic side effects. Embodiments of the present disclosure can address the problems and needs in the art that have not yet been fully solved by currently available Mtb treatments. Disclosed herein are a class of inhibitors of Hydrolase Important for Pathogenesis (Hip1; also known as Rv2224c). The disclosed inhibitors can address one or more of the above-discussed shortcomings in the art. Also disclosed herein is an assay (e.g., a lateral flow assay (LFA)) that incorporates one or more of the disclosed Hip1 inhibitors and is effective for selectively inhibiting contaminating, endogenous proteases. The disclosed assay can provide rapid, point-of-care testing effective for the detection of Mycobacterium tuberculosis.

Disclosed herein are potent inhibitor compounds targeting Hip1 and novel cocrystal structures of the potent Hip1-directed inhibitor compounds herein bound to Hip1. Hip1 is an Mtb serine hydrolase/protease, which has emerged as a promising drug target for the development of novel TB drugs. Hip1 is an Mtb cell wall-associated serine hydrolase that plays an important role in the pathogenic strategies of Mtb cell envelope maintenance and the dampening of host cell proinflammatory responses. Functional studies identify Hip1 as a target for drug discovery. Mice infected with a Hip1 transposon mutant strain survive significantly longer than wild-type Mtb-infected mice and exhibit mild lung immunopathology despite high bacterial burdens.

The disclosed Hip1 inhibitor compounds, including Inhibitor Compound 1 (696.72 g/mole) and Inhibitor Compound 2 (737.73 g/mole), have a relatively low molecular weight and are potent inhibitors of the drug target Hip1, with Ki values of 92±2 pM and Ki=117±15 pM, respectively. To date, no other drug-like, tight binding inhibitors target Hip1. As a result, antibiotic resistance has not evolved against Hip1-directed therapeutics.

The Hip1 inhibitor compounds disclosed herein comprise a tripeptide targeting sequence that directs the compound to the active site of Hip1 and a C-terminus modified to form an electrophilic warhead. The warhead can be configured to inactivate the Hip1 enzyme. In some embodiments, the C-terminus forms an alpha-keto alkyl ester, such as an alpha-keto methyl ester or an alpha-keto ethyl ester, to function as the electrophilic warhead.

### BRIEF DESCRIPTION OF THE DRAWINGS

FIG. 1 depicts an embodiment of the structure of an inhibitor compound 1 in accordance with one or more embodiments of the present disclosure, as determined by Nuclear Magnetic Resonance (NMR).

FIG. 2A is a line graph depicting the results of a direct killing assay showing the efficacy of the inhibitor compound 1 against Mycobacterium tuberculosis grown in liquid culture in accordance with one or more embodiments of the present disclosure;

FIG. 2B is a line graph depicting the inhibition of intracellular growth of Mycobacterium tuberculosis in its host cell, the macrophage, by inhibitor compound 1 with an IC₅₀=6.3±1.1 μM in accordance with one or more embodiments of the present disclosure;

FIG. 2C is a dot graph showing the inhbiitor compound 1 exhibits minimal cytotoxicity with RAW macrophages in accordance with one or more embodiments of the present disclosure;

FIG. 2D is a dot graph showing that the inhibitor compound 1 exhibits minimal cytotoxicity with HepG2 hepatocytes in accordance with one or more embodiments of the present disclosure;

FIG. 3A depicts the 2.7 Å X-Ray cocrystal structure of an embodiment of the inhibitor compound 1 (green sticks) herein covalently bound in the active site of Hip1 in accordance with one or more embodiments of the present disclosure;

FIG. 3B depicts well defined electron density (light blue chicken-wire) for an embodiment of the inhibitor compound 1 (green sticks) covalently bound to the active site serine 228 of Hip1 in accordance with one or more embodiments of the present disclosure;

FIG. 3C depicts van der Waals interactions that stabilize an embodiment the inhibitor compound 1 in the active site cleft of Hip1 in accordance with one or more embodiments of the present disclosure;

FIG. 3D depicts polar interactions that stabilize an embodiment of the inhibitor compound 1 in the active site cleft of Hip1 in accordance with one or more embodiments of the present disclosure;

FIG. 4 shows the line structure for an embodiment of the novel Hip1 chromogenic substrate as determined by NMR in accordance with one or more embodiments of the present disclosure;

FIG. 5 depicts an embodiment of fluorescent substrates for the detection of Hip1 enzymatic activity in accordance with one or more embodiments of the present disclosure;

FIG. 6 shows UFNG-2 is the superior substrate for M. bovis in cell culture in accordance with one or more embodiments of the present disclosure;

FIG. 7 depicts an embodiment of the cocrystal structure of Hip1 bound with Inhibitor Compound 2 in accordance with one or more embodiments of the present disclosure;

FIG. 8A depicts a prototype design for an embodiment of novel, competitive, lateral flow assay showing a positive result in accordance with one or more embodiments of the present disclosure;

FIG. 8B depicts a prototype design for an embodiment of a novel, competitive, lateral flow assay showing a negative result in accordance with one or more embodiments of the present disclosure;

FIG. 9 is a bar graph depicting M. bovis cells cleaving the fluorescent substrate UFNGlin a Hip1-dependent fashion in accordance with one or more embodiments of the present disclosure;

FIG. 10A is a line graph depicting Kₘ determinations for UFNG1 (17.5+4.4 μM) in accordance with one or more embodiments of the present disclosure;

FIG. 10B is a line graph depicting Kₘ determinations for UFNG2 (37.6+5.9 μM) in accordance with one or more embodiments of the present disclosure;

FIG. 11A is a reaction well picture showing the cleavage of the novel, chromogenic, substrate Cbz-Phe-Lys-Leu-pNa by M. bovis cells in accordance with one or more embodiments of the present disclosure;

FIG. 11B is a dot graph showing the cleavage of the novel, chromogenic, substrate Cbz-Phe-Lys-Leu-pNa by M. bovis cells in accordance with one or more embodiments of the present disclosure;

FIG. 12 is a table showing the three independent Kₘ determinations averaged for FIG. 13 in accordance with one or more embodiments of the present disclosure;

FIG. 13 is a line graph depicting a Kₘ determination for the novel, chromogenic, substrate Cbz-Phe-Lys-Leu-pNa in a Michaelis-Menten plot in accordance with one or more embodiments of the present disclosure;

FIG. 14A depicts an omit map for the X-Ray cocrystal structure of an embodiment of the inhibitor compound 2 (orange sticks) bound to Hip1, with the electron density shown as blue mesh according to the present disclosure;

FIG. 14B depicts an embodiment according to the present disclosure of the active site of Hip1 (grey sticks) bound with inhibitor compound 2 with labelled binding pockets S1 to S3, showing polar interactions as black dashed lines;

FIG. 14C depicts an embodiment according to the present disclosure of van der Waals interactions, shown as red dashed lines, with labelled binding pockets S1′ to S4 and water molecules as red spheres;

FIG. 15 depicts a superposition of Inhibitor Compound 1 and Inhibitor Compound 2.

FIG. 16A is a bar graph depicting M. bovis cleavage of the fluorescent substrate, UFNG2, in accordance with one or more embodiments of the present disclosure;

FIG. 16B is a bar graph depicting M. bovis cleavage of the chromogenic substrate, Cbz-Phe-Lys-Leu-pNa, in accordance with one or more embodiments of the present disclosure;

FIG. 17A is a bar graph showing that Inhibitor Compound 1 does not have significant cytotoxicity against THP1 macrophages.

FIG. 17B is a bar graph showing that Inhibitor Compound 2 does not have significant cytotoxicity against THP1 macrophages.

FIG. 17C is a bar graph showing Inhibitor Compound 1 effectively reduces MTB survival in THP1 macrophages.

FIG. 17D is a bar graph showing Inhibitor Compound 2 effectively reduces MTB survival in THP1 macrophages.

FIG. 18A depicts time-dependent antitubercular activity in THP-1 macrophages.

FIG. 18B depicts the average of the Hill slope values corresponding to the IC₅₀ values used to plot the mean IC₅₀ in FIG. 18A.

### DETAILED DESCRIPTION

With a global market in billions of dollars, FDA approved protease inhibitors have been very successful in treating a number of diseases including the Human Immunodeficiency Virus, type 2 diabetes, hepatitis, and obesity, to name a few. One drug development strategy is to design a compound comprising a moiety that targets the compound to the protease drug target. Conjugated to the targeting moiety is a “warhead” that inactivates the enzyme. An example of this approach is the FDA approved protease inhibitor, Bortezomib (Velcade®; Takeda) which is indicated for the treatment of multiple myeloma and mantle cell lymphoma. It contains a peptidomimetic targeting sequence conjugated to a boronic acid “warhead” that inactivates the catalytic threonine residue of its target, the 26 S protease.

### Example Hip1 Inhibitor Compounds

Disclosed herein are potent inhibitor compounds targeting Hip1 and novel enzyme complexes (e.g., cocrystal structures) of the potent Hip1-directed inhibitor compounds herein bound to Hip1. Hip1 is an Mtb serine hydrolase/protease, which has emerged as a promising drug target for the development of novel TB drugs. Hip1 is an Mtb cell wall-associated serine hydrolase that plays an important role in the pathogenic strategies of Mtb cell envelope maintenance and the dampening of host cell proinflammatory responses. Functional studies identify Hip1 as a target for drug discovery. Mice infected with a Hip1 transposon mutant strain survive significantly longer than wild-type Mtb-infected mice and exhibit mild lung immunopathology despite high bacterial burdens.

A compound for inhibiting a Hydrolase important for pathogenesis (Hip1) enzyme, the compound comprising the formula Prot-(X)ₙ—Z—(X)ₘ—Y, wherein “Prot” is an optional N-terminal protecting group, Z is a lysine or norarginine, each X is independently any amino acid or amino acid derivative, n is a number ranging from 1 to 10, m is a number ranging from 1 to 10, and Y is (i) a reporter group, or (ii) CO—CO₂R, wherein R is an alkyl, such that CO—CO₂R forms an alpha-keto alkyl ester. Additional examples of such Hip1 inhibitor compounds are described in more detail below.

In one or more embodiments of the present disclosure, the molecular scaffold comprises the amino acid sequence shown in Formula I:

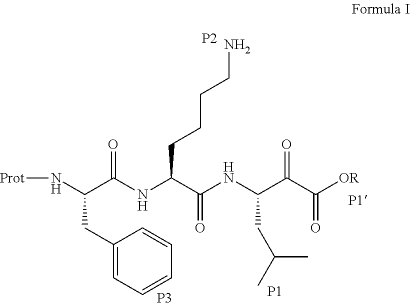

In Formula I, the amino acid at P3 is a phenylalinine (Phe), at P2 is a lysine (Lys), and at P1 is a leucine (Leu). The optional N-terminus protecting group (Prot) can form any suitable N-terminal protecting group known in the art. Examples include a pyrazine or carbamate-forming protecting group, such as benzyloxycarbonyl (Cbz), tert-butyloxycarbonyl (Boc), and fluorenylmethyloxycarbonyl (Fmoc). In all embodiments disclosing an N-terminal protecting group, the protecting group is optional. P1′ represents the warhead portion of the compound, in which the R group can be any suitable alkyl, such as a methyl or ethyl. Accordingly, a Hip1 inhibitor compound of the present disclosure can comprise an alpha-keto alkyl ester, such as an alpha-keto C1 to C6 alkyl ester, such as an alpha-keto methyl ester or an alpha-keto ethyl ester. As used herein, an “alkyl” includes both linear and branched isomers.

Various derivatives of Formula I are also included in the present disclosure. Example derivatives are shown in Table 1, below. Table 1 shows that the P1 residue can be substituted by a glutamine (Gln), the P2 residue can be substituted by a norarginine (nArg) (an arginine derivative with a side chain shortened by a single methylene group), and/or the P3 residue can be substituted by a tyrosine (Tyr). Each of the disclosed substitutions can be made independently. That is, any combination of the P3, P2, P1, P1′, and/or protecting group substitutions shown in Table 1 may be included in a Hip1 inhibitor compound of the present disclosure.

TABLE 1

| Name | Protecting Group | P3 | P2 | P1 | P1′ (warhead) |
|---|---|---|---|---|---|
| Inhibitor | Cbz | phenylalanine | lysine | leucine | alpha-keto methyl |
| Compound 1 | (benzyloxycarbonyl) | | | | ester |
| P1 derivative | Cbz | phenylalanine | lysine | glutamine | alpha-keto methyl |
| | (benzyloxycarbonyl) | | | | ester |
| P2 derivative | Cbz | phenylalanine | norarginine | leucine | alpha-keto methyl |
| | (benzyloxycarbonyl) | | | | ester |
| P3 derivative | Cbz | tyrosine | lysine | leucine | alpha-keto methyl |
| | (benzyloxycarbonyl) | | | | ester |
| Warhead derivative | Cbz | phenylalanine | lysine | leucine | alpha-keto ethyl |
| | (benzyloxycarbonyl) | | | | ester |
| Protecting Group | pyrazine amide | phenylalanine | lysine | leucine | alpha-keto methyl |
| derivative | | | | | ester |

In one or more embodiments, the protecting group of the compound comprises benzyloxycarbonyl (Cbz). In one or more embodiments, the P3 residue of the compound comprises phenylalanine (Phe) or tyrosine (Tyr). The P2 residue of the compound may comprise lysine (Lys) or norarginine (nArg). The P1 residue of the compound may comprise leucine (Leu) or glutamine (Gln). In one or more embodiments, the warhead comprises an alpha-keto alkyl ester, such as an alpha-keto methyl ester (—CO—CO₂Me) or an alpha-keto ethyl ester (—CO—CO₂Et).

In one or more embodiments, the molecular scaffold comprises the amino acid sequence as shown in Formula II:

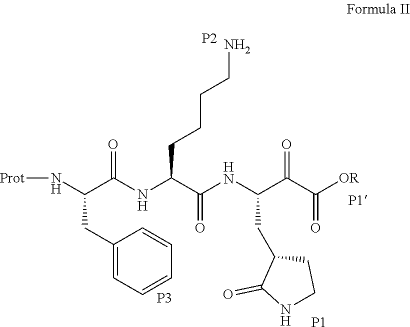

In Formula II, the amino acid at P3 is a phenylalanine (Phe), at P2 is a lysine (Lys), and at P1 is a glutamine lactam. The optional N-terminus protecting group (Prot) and the R group are defined as in Formula I.

Various derivatives of Formula II are also included in the present disclosure. Example derivatives are shown in Table 2, below. Table 2 shows that the P1 residue can be substituted by a norleucine (nLeu) (a leucine derivative with an n-butyl side chain rather than an isobutyl side chain). As with Formula I, the P2 residue can be substituted by a norarginine (nArg) and/or the P3 residue can be substituted by a tyrosine (Tyr). Each of the disclosed substitutions can be made independently. That is, any combination of the P3, P2, P1, P1′, and/or protecting group substitutions shown in Table 2 may be included in a Hip1 inhibitor compound of the present disclosure.

TABLE 2

| Name | Protecting Group | P3 | P2 | P1 | P1′ (warhead) |
|---|---|---|---|---|---|
| Inhibitor | Cbz | phenylalanine | lysine | glutamine | alpha-keto |
| Compound 2 | (benzyloxycarbonyl) | | | lactam | methyl ester |
| P1 derivative | Cbz | phenylalanine | lysine | norleucine | alpha-keto |
| | (benzyloxycarbonyl) | | | | methyl ester |
| P2 derivative | Cbz | phenylalanine | norarginine | glutamine | alpha-keto |
| | (benzyloxycarbonyl) | | | lactam | methyl ester |
| P3 derivative | Cbz | tyrosine | lysine | glutamine | alpha-keto |
| | (benzyloxycarbonyl) | | | lactam | methyl ester |
| Warhead derivative | Cbz | phenylalanine | lysine | glutamine | alpha-keto |
| | (benzyloxycarbonyl) | | | lactam | ethyl ester |
| Protecting Group | pyrazine amide | phenylalanine | lysine | glutamine | alpha-keto |
| derivative | | | | lactam | methyl ester |

In one or more embodiments, the protecting group comprises Cbz. In one or more embodiments, the P3 residue of the compound comprises Phe or Tyr. In one or more embodiments, the P2 residue of the compound comprises Lys or nArg. The P1 derivative of the compound may comprise Prot-Phe-Lys-Glutamine lactam (Glnlac)-CO—CO₂R. In one or more embodiments, the P1 residue comprises Glnlac or norleucine (nLeu). As in Formula I, the warhead of the compound may include an alpha-keto alkyl ester such as —CO—CO₂Me or —CO—CO₂Et.

Other substitutions can also be made (e.g., to either Formula I or Formula II) to form alternative Hip1 inhibitor compounds. For example, P3 and/or P1 can be substituted such that the compound comprises Prot-X-Lys-X—CO—CO₂R, where X is any amino acid or amino acid derivative (where each separate X amino acid can be selected independently), and Prot and R are defined as above in Formulas I and II.

In one or more embodiments, a Hip1 inhibitor compound can comprise Prot-Xₙ-Lys-Xₘ—CO—CO₂R, where n is any whole number (e.g., 1 to 10 or 1 to 8 or 1 to 6 or 1 to 4) and indicates that there may be any such whole number of X residues to the N-terminus side of the lysine, m is any whole number (e.g., 1 to 10 or 1 to 8 or 1 to 6 or 1 to 4) and indicates that there may be any such whole number of X residues to the C-terminus side of the lysine, and X, Prot, and R are defined as above (and where each separate Xₙ and/or Xₘ amino acid can be selected independently).

Truncated derivatives may comprise Prot-Leu-CO—CO₂R, Prot-X—CO—CO₂R, Prot-Lys-Leu-CO—CO₂R, or Prot-Lys-X—CO—CO₂R, where X, Prot, and R are defined as above. N-terminal lengthened derivatives may comprise Prot-Xₙ-Phe-Lys-Leu-CO—CO₂R, where X, n, and R are defined as above. In some embodiments, there are no glycine residues, in the C-terminal direction, within two residues from the lysine (or norarginine) of the targeting sequence.

As used herein, an “amino acid derivative” refers to an amino acid (proteinogenic or non-proteinogenic) that includes a modified side chain and/or terminal. Modifications can include, for example, lengthening or shortening of the side chain (e.g., by 1 or 2 carbon atoms), the addition of a hydroxyl group (i.e., hydroxylation) to a carbon atom of the amino acid, the addition of a carboxylate group (i.e., carboxylation) to a carbon atom of the amino acid, the addition of a phosphate (i.e., phosphorylation) to a heteroatom (such as an O atom) of the amino acid, the attachment of a sulfur-based group (e.g., a sulfo group) to a heteroatom (such as an O or S atom) of the amino acid, the addition of a carbohydrate (i.e., glycosylation) to a heteroatom (such as an N or O atom) of the amino acid, the addition of an alkyl group (e.g., a C1 to C6 alkyl) to a heteroatom (such as an N, O, or S atom) of the amino acid, or the addition of an acyl group (i.e., acylation) to a heteroatom (such as an N, O, or S atom) of the amino acid. Amino acid derivatives in which the side group is extended can form ring structures (e.g., lactam rings) in the side group. Conversely, derivatives of amino acids that include ring structures (phenylalanine, tyrosine, tryptophan) can lose one or more carbon atoms resulting in opening of the corresponding ring structure.

The electrophilic warhead can include any of the functional groups disclosed herein as suitable warheads (e.g., alpha-keto alkyl esters) and can additionally or alternatively include any electrophilic group capable of forming a covalent attachment to active site Ser228 of Hip1.

Additionally provided herein are methods for purification of Hip1 and methods for co-crystallization of Hip1 bound with a Hip1 inhibitor compound as disclosed herein. Accordingly, disclosed embodiments include such methods as well as the X-ray cocrystal structure of Hip1 bound with a Hip1 inhibitor compound as disclosed herein. Example Hip1 inhibitor compounds include those referred to herein as “Inhibitor Compound 1” (which is an example of a compound of Formula I) and “Inhibitor Compound 2” (which is an example of a compound of Formula II) as well as other Hip1 inhibitor compounds disclosed herein.

### Example Probes & Assays

Introducing a point-of-care (POC) diagnostic assay in the treatment of TB can lead to more efficient testing and appropriate care for a pathogen as transmissible as Mtb. POC assays are critical for achieving the EndingTB-2030 objective, which is an initiative of the World Health Organization. POC diagnostic assays provide results immediately to the patient without costly laboratory work. The use of a POC diagnostic assay is crucial in providing accurate and rapid results to individuals infected with Mtb so they can get adequate care. Currently, about one-third of TB patients go undiagnosed. In Africa, up to 50% of TB patients are believed to go undiagnosed.

In one or more embodiments, a novel substrate comprises Prot-Phe-Lys-Leu-pNa-(paranitroanilide) or Prot-Phe-Lys-Gln-pNa or derivatives of these compounds, including those conjugated to (in addition to or alternative to pNa) a chromophore, fluorophore, antibody, nanobody (e.g., nanoparticle), and/or other reporter group. Such compounds can be used as molecular probes in an enzymatic activity assay, serine protease purification method, serine/threonine protease detection assay, and/or rapid diagnostic test for the presence of Mtb.

The disclosed assays may be configured to test for the presence of Mtb in samples of patient blood, sputum, and/or other body fluids. For example, rapid diagnosis of active TB in patients (e.g., those who have TB but show negative sputum smears for acid fast bacteria) may enable prompt, highly accurate identification of drug-resistant strains of M. tuberculosis. The disclosed compounds can function as molecular probes useful for the detection of Mtb in patients.

For example, an ELISA assay may include a Hip1 inhibitor compound as disclosed herein. If Mtb is present in the patient's sputum, the inhibitor compound will bind to Hip1 on the surface of Mtb. A secondary antibody can then be added to generate a detectable signal. Variations of this assay may include additional binding systems, for exampe the biotin-streptavidan system.

Further provided herein is a bait substrate for the detection of Hip1. Such bait substrates may be utilized in a detection assay, such as a lateral flow assay. Lateral flow assays, a type of POC diagnostic assay, have been developed for several small molecules. Example LFA's on the market include pregnancy tests, COVID-19 tests, and assays that detect the presence of illicit drugs. LFAs can be designed as competitive assays or sandwich assays. It is often difficult to develop two antibodies to bind a small molecule analyte, which is required for the development of a sandwich-based LFA. Thus, competitive assays are often preferred for the detection of small molecules. Provided herein is a novel, lateral flow diagnostic assay for the detection of Mtb. The disclosed assay can be designed in the competitive assay format. Modification of the assay may be employed to develop a sandwich-based assay. The present disclosure also includes other Hip1-dependent substrate cleavage immunoassays (i.e. ELISA assay, Western Blot assay, etc.) for the detection of Mtb.

The bait substrate can include (X)ₙ—P₃—P₂—P₁—(X)ₘ, wherein: X is any amino acid, amino acid derivative, or hapten; n is any whole number (e.g., 1 to 10 or 1 to 8 or 1 to 6 or 1 to 4); m is any whole number (e.g., 1 to 10 or 1 to 8 or 1 to 6 or 1 to 4); P₃ is phenylalanine or tyrosine; P₂ is lysine or norarginine; and P₁ is leucine, glutamine, glutamine lactam, or norleucine. The bait substrate can include Xₙ-Phe-Lys-Leu-Xₘ or Xₙ-Phe-Lys-Glnlac-Xₘ. Example haptens include keyhole limpet hemocyanin, bovine serum albumin, and ovalbumin. In some embodiments where a hapten is included, the hapten is disposed at the N-terminus and/or C-terminus. That is, the compound omits residues that are farther C-terminal or farther N-terminal of the hapten. In some embodiments, Xₙ and/or Xₘ are omitted (i.e., n and/or m equal 0). Example subset compounds include Xₙ₊₁-Phe-Lys-Leu-Xₙ₋₁ and Xₙ₊₁-Phe-Lys-Glnlac-Xₙ₋₁.

Example derivatives of Inhibitor Compound 1 and Inhibitor Compound 2 useful as bait substrates are shown in Tables 3 and 4, respectively. Each of the disclosed substitutions can be made independently. That is, any combination of the substitutions shown in Table 3 or Table 4 may be included in a bait substrate compound of the present disclosure.

TABLE 3

| Name | P4 | P3 | P2 | P1 | P1′ |
|---|---|---|---|---|---|
| Inhibitor | Cbz | phenyl- | lysine | leucine | Alpha-keto |
| Compound 1 | | alanine | | | alkyl ester |
| | Xₙ | phenyl- | lysine | leucine | Xₘ |
| | | alanine | | | |
| | Xₙ | phenyl- | lysine | glutamine | Xₘ |
| | | alanine | | | |
| | Xₙ | phenyl- | norar- | leucine | Xₘ |
| | | alanine | ginine | | |
| | Xₙ | Tyrosine | lysine | leucine | Xₘ |

TABLE 4

| Name | P4 | P3 | P2 | P1 | P1′ |
|---|---|---|---|---|---|
| Inhibitor | Cbz | phenyl- | lysine | glutamine | Alpha-keto |
| Compound 2 | | alanine | | lactam | alkyl ester |
| | Xₙ | phenyl- | lysine | norleucine | Xₘ |
| | | alanine | | | |
| | Xₙ | phenyl- | norar- | glutamine | Xₘ |
| | | alanine | ginine | lactam | |
| | Xₙ | tyrosine | lysine | glutamine | Xₘ |
| | | | | lactam | |
| | Xₙ | phenyl- | lysine | glutamine | Xₘ |
| | | alanine | | lactam | |

In one or more embodiments, an assay for the detection of Hip1 comprises the bait substrate and a detection antibody (or immobilized Hip1) with or without conjugated colloidal gold nanoparticles and/or other detection component. Also disclosed is an assay kit for the detection of Hip1 comprising the assay herein and assay reagents.

Further provided herein is a composition and method for selectively inhibiting endogenous proteases in a physiological sample (e.g., a sputum sample). In certain embodiments the method comprises providing a protease inhibitor cocktail wherein the cocktail comprises one or more of EDTA (metalloprotease inhibitor), phenylmethylsulfonyl fluoride (PMSF; serine protease inhibitor), E-64 (cysteine protease inhibitor), and pepstatin A (aspartyl protease inhibitor) and incubating the sample (e.g., a sputum sample, a blood sample, or other body fluid sample from a patient) with the protease inhibitor cocktail prior to testing. The protease inhibitor cocktail can function to inhibit endogenous proteases that could proteolytically cleave the bait substrate, potentially leading to a false positive result. The one or more protease inhibitors included in the protease inhibitor cocktail preferably inhibit endogenous proteases with significantly greater inhibition than any incidental inhibition of Hip1.

The inhibitor compounds disclosed herein may be used as molecular probes useful for the discovery of other protease drug targets for Tuberculosis, as well as probes for serine/threonine proteases involved in the pathologies of other diseases.

### Vaccine Component

The disclosed Hip1 inhibitor compounds can be used as a vaccination component, such as a booster to the BCG vaccine. For example, the Hip1 inhibitor compounds may be used as a booster for the bacilli Calmette-Guerin (BCG) vaccine which is has variable efficacy (0-80%) in preventing pulmonary TB. Since Hip1 plays a role in suppression of the host's immune response, the disclosed inhibitor compound may serve to boost a patient's immune response when given in concert with the BCG vaccine, thus increasing the efficacy of the vaccine.

### Performance Characteristics of Example Hip1 Inhibitor Compounds

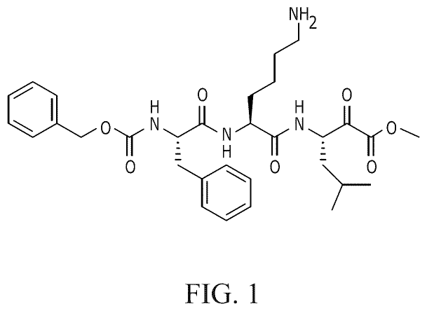

FIG. 1 is a molecular diagram line drawing depicting the scaffold structure of an example embodiment “Inhibitor Compound 1”. The illustrated inhibitor compound is a potent inhibitor of Hip1. In one or more embodiments, the inhibitor compounds contain a peptide targeting sequence that directs the compound to the active site of Hip1. The peptide targeting sequence may include the tripeptide Phe-Lys-Leu, as shown in FIG. 1. A C-terminal alpha-keto methyl ester or other electrophilic warhead may be conjugated to the targeting sequence. The warhead acts to quiesce the activity of the active site serine 228 residue of the enzyme, thus rendering the drug target inactive.

Advantages of these compounds include their potency for Hip1 (Inhibitor Compound 1, Ki=92+15 pM and Inhibitor Compound 2, Ki=117+15 pM), low molecular weight (Inhibitor Compound 1:696.72 g/mole and Inhibitor Compound 2:737.73 g/mole), and its drug target, Hip1. To date, no other existing drug-like, tight binding inhibitors target Hip1. As a result, antibiotic resistance has not evolved against Hip1-directed therapeutics. Additionally, inhibition of Hip1 may boost the host's immune response to help clear the infection. No other anti-TB drug on the market uses this strategy to promote bacterial clearance. Furthermore, since Hip1 plays an important role in Mtb cell envelop maintenance, using Inhibitor Compound 1 in conjunction with current FDA approved antibiotics may result in greater bacterial clearance efficacy.

According to a recent report a Mtb transposon mutant of Hip1 exhibits severe growth attenuation in the presence of 0.5 μg/mL of ethambutol, which affected the growth of WT Mtb only marginally. This mutant also showed increased sensitivity to the antibiotics meropenem, vancomycin, and rifampicin. This indicates that inhibition of Hip1 may result in reduced Mtb fitness at partial inhibitory antibiotic concentrations. Therefore, such Mtb treatment regimens may require lower concentrations of antibiotics and/or a reduced treatment time, potentially increasing patient compliance and reducing toxic side effects. It should be noted that the use of multiple drugs (combination therapy) is a very successful approach in treating a number of bacterial, viral, fungal, and parasitic infections.

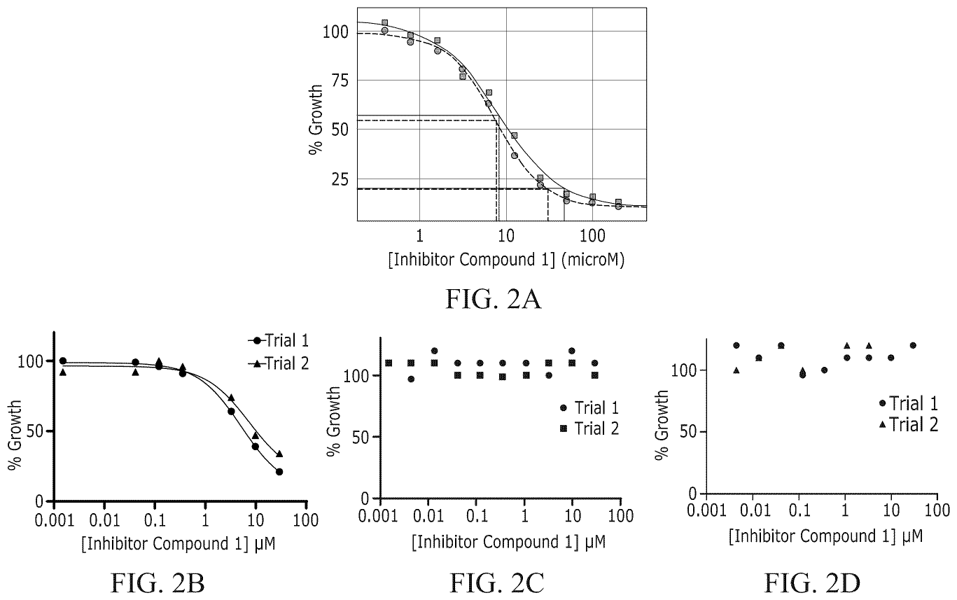

FIG. 2A depicts the results of a direct killing assay showing the efficacy of an embodiment of the inhibitor against Mtb grown in liquid culture according to one or more embodiments of the present disclosure, with the black boxes and circles showing two separate experimental trials. The inhibitor herein kills Mtb with an IC50=8.9+/−1.7 μM. In the illustration the circles and squares represent the results of two independent trials under identical conditions. The illustrated inhibitor compound is a potent inhibitor of Hip1.

In addition to high potency and low molecular weight, the inhibitor compound herein can function as a reversible inhibitor. Reversible inhibitors typically exhibit lower toxicity in the case of off-target binding.

FIG. 2B is a line graph showing the growth inhibition of Mtb when tested in an intracellular RAW macrophage assay, with triangles and circles showing two different experimental trials. RAW macrophages were infected with Mtb overnight and then treated with the inhibitor compound at various concentrations for seven days at which time the cytotoxicity of the inhibitor on Mtb was assessed, as previously mentioned. The inhibitor has an IC50=6.3+1.1 μM.

FIG. 2C is a dot graph depicting the results of RAW macrophages dosed with various concentrations of the inhibitor, with the black boxes and circles showing two different experimental trials. Importantly, the compound shows minimal cytotoxicity towards RAW macrophages, with a TC50 value >100 μM.

FIG. 2D is a plot of HepG2 hepatocytes versus the concentration of an embodiment the inhibitor herein, with the black triangles and circles showing two different experimental trials. Importantly, the compound shows minimal cytotoxicity towards HepG2 hepatocytes, with a TC50 value >100 μM.

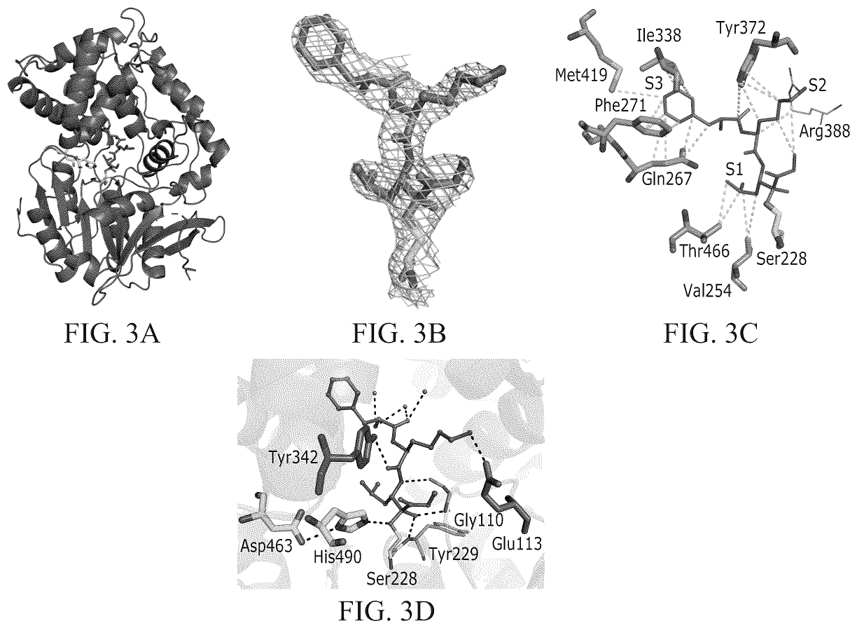

FIGS. 3A to 3D illustrate the three-dimensional atomic X-ray cocrystal structure of Hip1 bound with an embodiment of Hip1 Inhibitor Compound 1 herein. Inspection of the structure reveals that the inhibitor forms a covalent interaction with the active site Ser228 of Hip1, thus rendering the enzyme inactive. This constitutes a novel ligand binding mode in the active site of Hip1.

FIG. 3A illustrates the 2.7 Å cocrystal structure of Hip1 Inhibitor Compound 1. Hip1 is shown in ribbon form and the catalytic triad of Hip1 And Inhibitor Compound 1 shown in stick form.

FIG. 3B illustrates the well-defined electron density (chicken wire; σ level=1.0) for the inhibitor (sticks) covalently bound to the active site Ser228 (also as sticks) of Hip1, thus inactivating the enzyme. The absence of electron density for the Cbz protecting group suggests that it is disordered in the structure.

FIG. 3C illustrates the cocrystal structure of Hip1 bound with Inhibitor Compound 1. Amino acid resides of Hip1 that make nonpolar interactions with Inhibitor Compound 1 are labelled around the periphery of Inhibitor Compound 1, with binding pockets labelled as S1, S2, and S3. Nonpolar, van der Waals interactions are shown as dotted lines.

FIG. 3D further illustrates the cocrystal structure of Hip1 Inhibitor Compound 1. Amino acid residues of Hip1 that make polar interaction with Inhibitor Compound 1 are labelled. Polar interactions are shown as dotted lines. Water molecules are shown as spheres. The salt bridge between P2 Lys of the inhibitor and the carboxylate of Glu113 demonstrates selectivity for Lys observed in substrate profiling experiments.

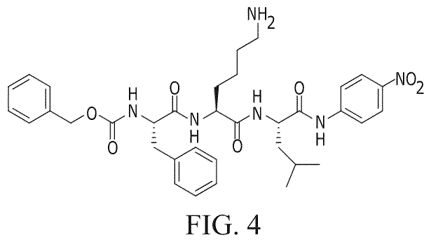

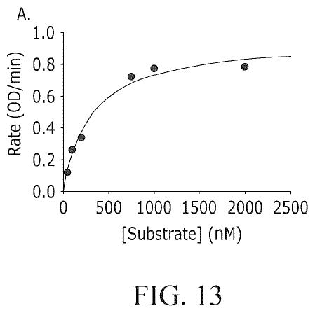

FIG. 4 illustrates the structure of an example substrate analogue derived from the inhibitor compound (Km=421+23 nM; see FIGS. 12 and 13). The illustrated compound includes a chromogenic para-nitroanilide (pNa) C-terminus. The novel chromogenic Hip1 substrate is useful for enzymatic characterization and detection of Hip1. Upon Hip1-dependent cleavage of the peptide bond between P1 leucine and the pNa group, there is an increase in absorbance at 405 nm as detected by a spectrophotometer or plate-reader. Rapid, field-ready assays may also benefit from the potency of the novel, chromogenic substrate Cbz-Phe-Lys-Leu-pNa (Km=421+23 nM) described herein. Upon Hip1-dependent cleavage of the Leu-pNa bond, there is an increase in absorbance at 405 nM.

Patient sputum samples positive for TB will contain Hip1, thus will react with the substrate to yield a change in absorbance which can be read on a portable spectrophotometer. Nonspecific cleavage of the substrate can be mitigated by the inclusion of protease inhibitors that do not inhibit Hip1 but do inhibit other proteases.

The LFA provided herein is based on the potent, Hip1 recognition sequences from the inhibitor compounds herein, which can be modified to generate a “molecular bait” substrate for the detection of Mtb via Hip1-dependent cleavage. Hip1 is particularly suitable as a reporter for the presence of Mtb because it resides on the cell surface of the bacterium, thus has access to the molecular bait substrate for peptide bond hydrolysis (cleavage). Modifying the C-terminal of the bait substrate to include any natural or non-natural amino acid or hapten (e.g. keyhole limpet hemocyanin, bovine serum albumin, or ovalbumin) can result in a peptide bond cleavable by Hip1.

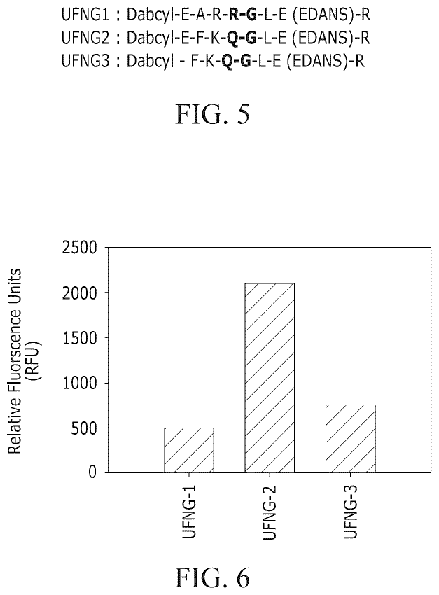

FIG. 5 depicts example fluorescent substrates for the detection of Hip1 enzymatic activity according to aspects of the present disclosure. Hip1 cleaves the peptide bond between the amino acids Arginine (R)-Glycine (G), and Glutamine (Q)-Glycine (G). In these examples, the fluorophore is EDANS (5-((2-Aminoethyl)amino) naphthalene-1-sulfonic acid) and the quencher is DABCYL (4-((4-(dimethylamino)phenyl) azo)benzoic acid). Other fluorophore and quencher pairs are known in the art and may be utilized.

FIG. 6 depicts results of M. bovis cleavage of example fluorescent substrates. M. bovis cells normalized by OD600 were incubated with 150 μM substrate for 2 hrs, 25 degrees C., with rotation.

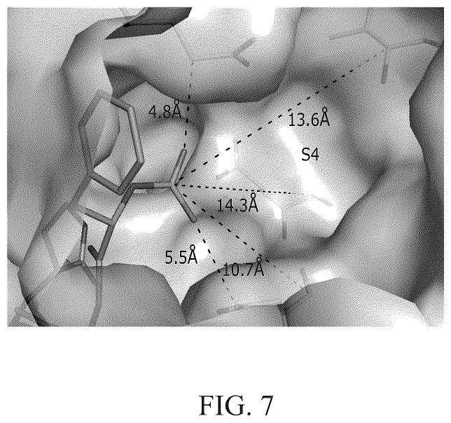

FIG. 7 depicts an embodiment of the cocrystal structure of Hip1 bound with Inhibitor Compound 2 and showing a large S4 pocket adequate to accommodate additional N-terminal moieties on a “molecular bait” substrate sequence.

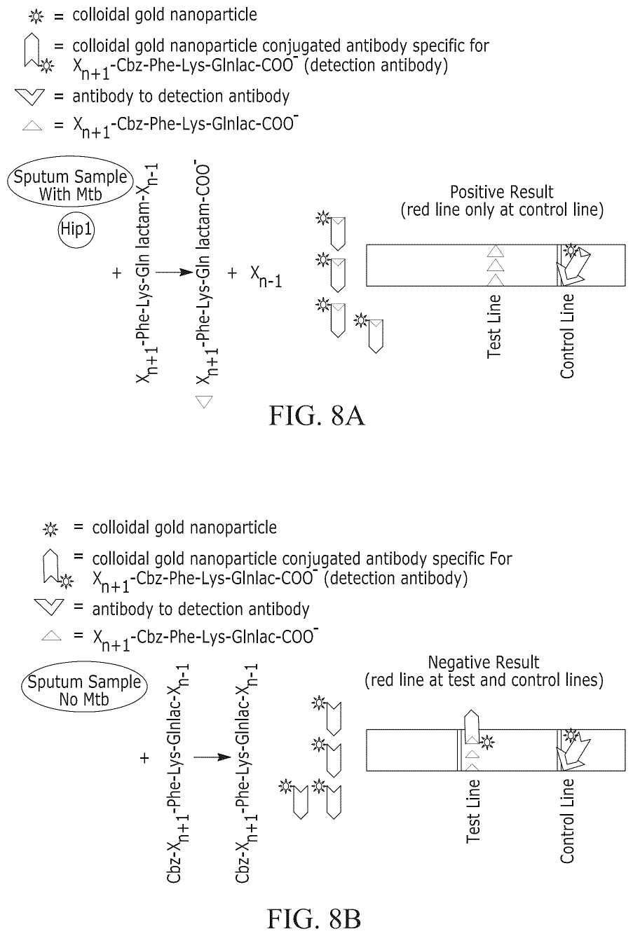

FIGS. 8A and 8B depict an embodiment of a competitive lateral flow assay showing a positive result for the presence of Mycobacterium tuberculosis, with FIG. 8A showing a positive result and FIG. 8B showing a negative result. Cleavage of the bait substrate (Xn+1-Phe-Lys-Glnlac-Xn−1) sequence by Hip1 unmasks a hidden C-terminal epitope (Xn+1-Phe-Lys-Glnlac-COO⁻) (also referred to herein as “the cleaved epitope”). The detection antibodies (mono- or polyclonal) are configured to bind to the Xn+1-Phe-Lys-Glnlac-COO epitope. Analysis of the cocrystal structure of Hip1 bound with Inhibitor Compound 2 reveals that there is ample room in the S4 pocket of the substrate binding site of Hip1 to lengthen the substrate (see FIG. 7), if required for epitope optimization. For example, six amino acids can be included to constitute a sufficient epitope.

Colloidal gold nanoparticles or a similar detection system, which generate a visible line on the test strip, may be conjugated to the detection antibody. In some embodiments, such as shown, Xn+1-Phe-Lys-Glnlactam-COO⁻ is immobilized at the test line. A capture antibody against the detection antibody may be immobilized at the control line, as shown.

If Mtb is present in the sputum sample, Hip1 cleaves the molecular bait substrate, Xn+1-Phe-Lys-Glnlac-Xn−1, and liberates the analyte, Xn+1-Phe-Lys-Glnlac-COO⁻, which in certain embodiments is recognized by the colloidal gold nanoparticle conjugated detection antibody. This prevents the binding of the detection antibody to the immobilized Xn+1-Phe-Lys-Gln lactam-COO⁻ at the test line, thus no visible line appears (or only a faint line appears) (FIG. 8A, positive result). Bound detection antibody may continue to migrate to the control line where an antibody against the detection antibody can be immobilized, thus resulting in a visible line indicating that the assay is functioning properly.

If Mtb is absent from the patient sample, then there is no Hip1-dependent proteolytic cleavage of the bait substrate, Xn+1-Phe-Lys-Gln lactam-Xn−1, thus the hidden epitope is not unmasked. Consequently, the detection antibody remains unbound and can bind to the immobilized Xn+1-Phe-Lys-Gln lactam-COO— at the test line resulting in a visible line (negative result, FIG. 8B). Unbound test line antibody continues migration to the control line where an antibody against the detection line antibody is immobilized, thus resulting in a visible line, which indicates the assay is functioning properly.

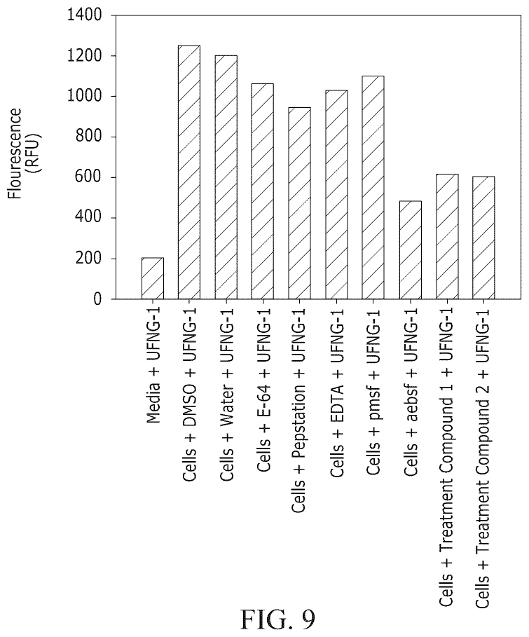

FIG. 9 is a bar graph depicting M. bovis cells cleaving the fluorescent substrate UFNG1 in a Hip1-dependent fashion.

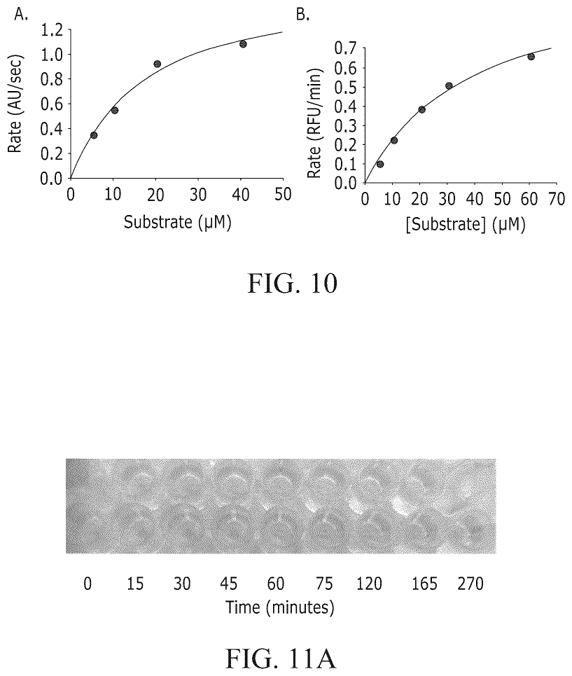

FIG. 10A is a line graph depicting Km determinations for UFNG1 (17.5+4.4 μM) in accordance with one or more embodiments of the present disclosure.

FIG. 10B is a line graph depicting Km determinations for UFNG2 (37.6+5.9 μM) in accordance with one or more embodiments of the present disclosure.

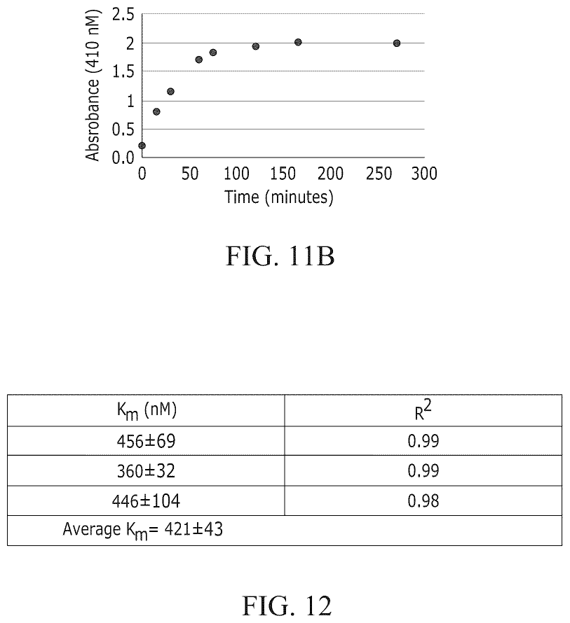

FIG. 11A is a reaction well picture showing the cleavage of the novel, chromogenic, substrate Cbz-Phe-Lys-Leu-pNa by M. bovis cells. FIG. 11B is a dot graph depicting experimental results showing the cleavage of the novel, chromogenic, substrate Cbz-Phe-Lys-Leu-pNa by M. bovis cells.

Derivatives of Inhibitor Compound 1 and Inhibitor Compound 2 can include a fluorophore and corresponding quencher attached thereto. Various fluorophore and quencher pairs are known in the art and may be utilized.

Forster Resonance Energy Transfer (FRET) substrates are cleaved by M. bovis cells, with UFNG2 being the most effective substrate in cell culture (see FIG. 6). Additionally, the substrate sequence may be lengthened on the N-terminus with any natural or non-natural amino acid(s), protecting group, or hapten (e.g. keyhole limpet hemocyanin, bovine serum albumin, or ovalbumin), as disclosed herein.

FIG. 12 is a table showing the three independent Km determinations averaged for the figure.

FIG. 13 is a line graph depicting a Km determination for the novel, chromogenic, substrate Cbz-Phe-Lys-Leu-pNa, according to one or more embodiments of the present disclosure, in a Michaelis-Menten plot.

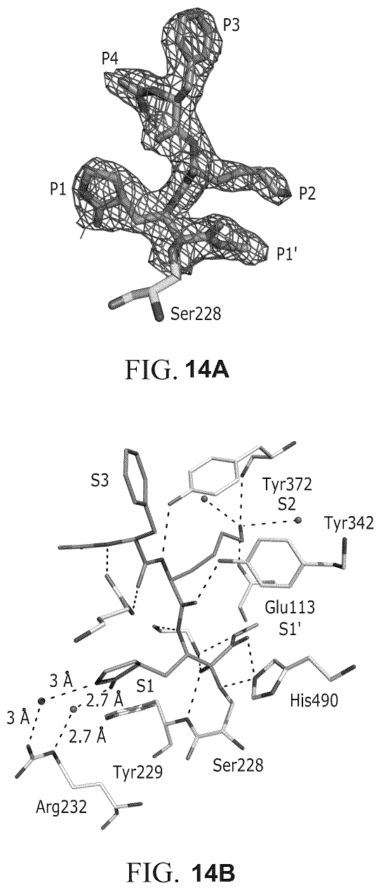

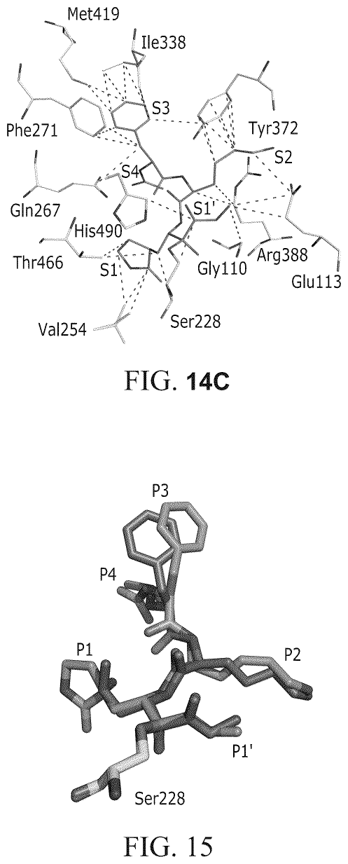

FIGS. 14A to 14C illustrate the cocrystal structure of Hip1 bound with Inhibitor Compound 2. FIG. 14A depicts the |(Fo)-(Fc)| difference density, shown as mesh and contoured at 1σ. The inhibitor compound and the catalytic Ser228 are rendered as sticks. FIG. 14B illustrates the polar interactions, shown as dashed lines, of the active site of Hip-1, with corresponding amino acid sites labelled and shown around the periphery of the inhibitor compound. The binding pockets are labeled S1, S2, and S3 and water molecules are shown as spheres. FIG. 14C depicts the van der Waals interactions, shown as dashed lines. The binding pockets for the inhibitor compound are labeled as S1′, S2, S3, and S4. FIG. 15 illustrates one embodiment of a superposition of Inhibitor Compound 1 and Inhibitor Compound 2.

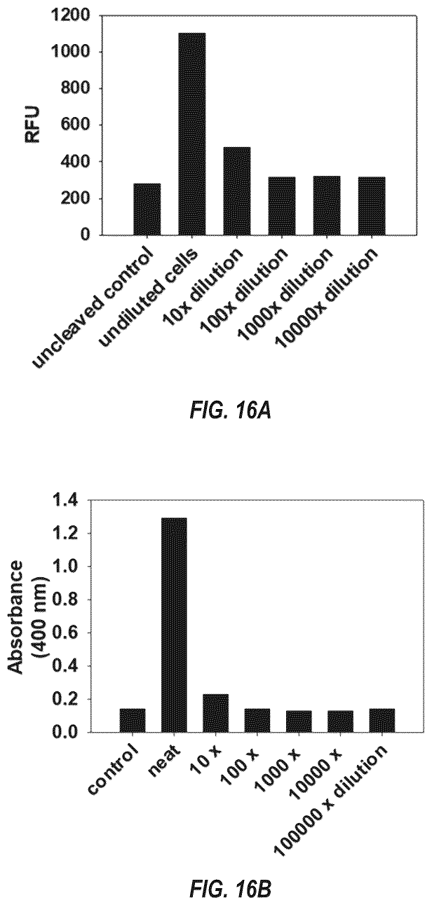

FIG. 16A is a bar graph depicting M. bovis cleavage of the fluorescent substrate, UFNG2, in accordance with one or more embodiments of the present disclosure. M. bovis cells were grown to log phase and then incubated with UFNG2 (260 μM) for two hours, with rotation at 25 degrees C. Serial dilutions of cells were made in M. bovis culture medium (7H9+OADC).

FIG. 16B is a bar graph depicting M. bovis cleavage of the chromogenic substrate, Cbz-Phe-Lys-Leu-pNa, in accordance with one or more embodiments of the present disclosure. M. bovis cells were grown to log phase then incubated with Cbz-Phe-Lys-Leu-pNa (260 μM) for two hours, with rotation at 25 degrees C. Serial dilutions of cells were made in M. bovis culture medium (7H9+OADC).

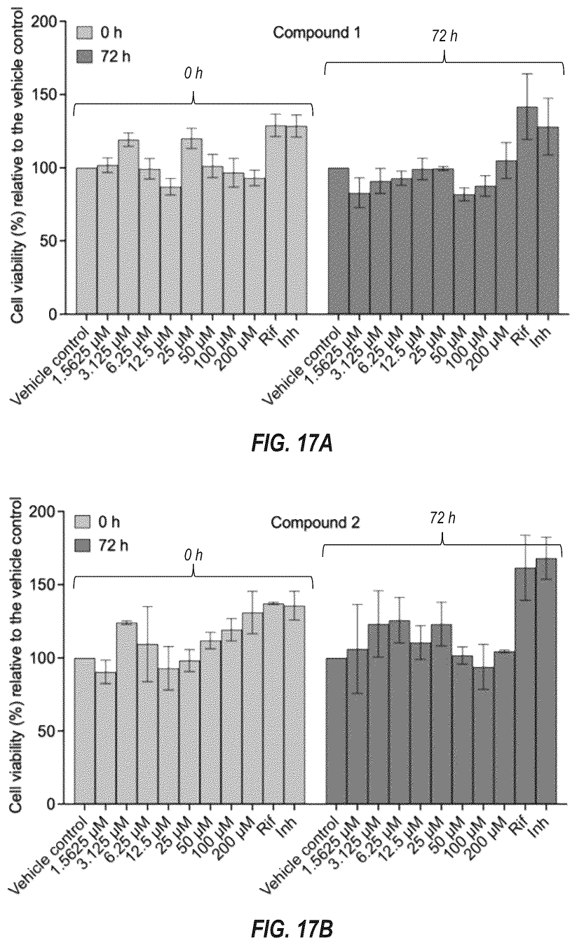

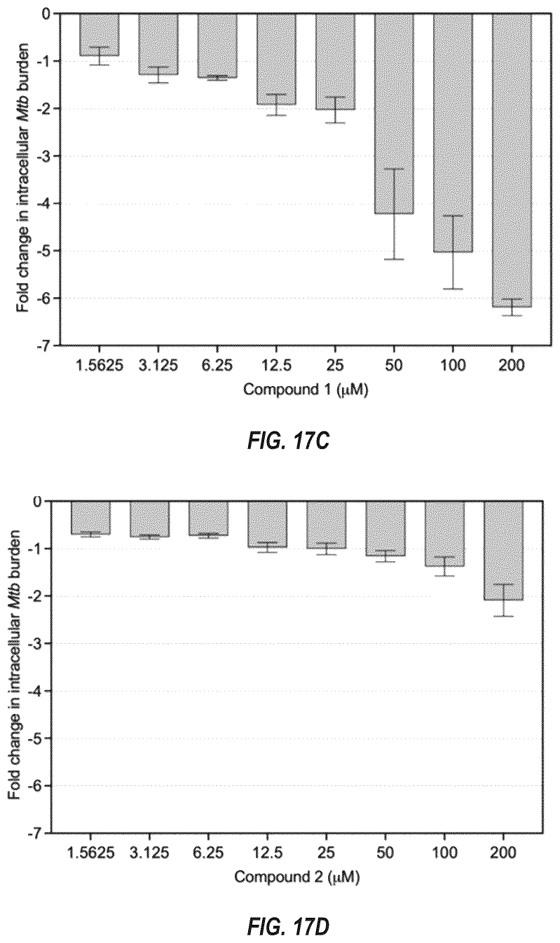

FIG. 17A is a bar graph showing that Inhibitor Compound 1 does not have significant cytotoxicity against THP1 macrophages. FIG. 17B is a bar graph showing that Inhibitor Compound 2 does not have significant cytotoxicity against THP1 macrophages. FIG. 17C is a bar graph showing that Inhibitor Compound 1 effectively reduces Mtb survival in THP1 macrophages. FIG. 17D is a bar graph showing that Inhibitor Compound 2 effectively reduces Mtb survival in THP1 macrophages.

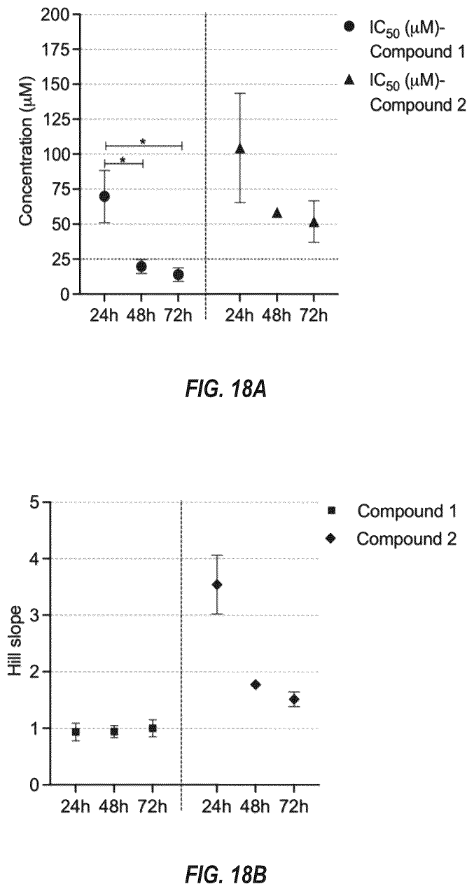

FIG. 18A depicts time-dependent antitubercular activity in THP-1 macrophages. FIG. 18B depicts the average of the Hill slope values corresponding to the IC₅₀ values used to plot the mean IC₅₀ in FIG. 18A. The IC₅₀ values for Inhibitor Compounds 1 and 2 are shown in tabulated form in Table 5, below.

TABLE 5

| | Compound 1 IC₅₀ | | Compound 2 IC₅₀ | |
|---|---|---|---|---|
| Time | Mean (μM) ± | | Mean (μM) ± | |
| Point | SEM | Range | SEM | Range |
| 24 h | 69.74 ± 18.65 | 51.09 to 8.38 | 104.41 ± 39.06 | 44.35 to 177.7 |
| 48 h | 19.64 ± 4.98 | 12.7 to 29.29 | 58.44 ± 1.15 | 57.29 to 59.59 |
| 72 h | 13.92 ± 4.86 | 8.237 to 23.6 | 51.79 ± 14.74 | 25.56 to 53.26 |

### Additional Terms & Definitions

Reference throughout this specification to “one embodiment,” “an embodiment,” or similar language means that a particular feature, structure, or characteristic described in connection with the embodiment is included in at least one embodiment of the present disclosure. Thus, appearances of the phrases “in one embodiment,” “in an embodiment,” and similar language throughout this specification may, but do not necessarily, all refer to the same embodiment.

Additionally, reference to “Inhibitor Compound 1” and “Inhibitor Compound 2” recognizes that in various embodiments the respective inhibitor functions as a treatment compound, a diagnostic compound, an assay compound, a testing compound for research, or in any other capacity involving an interaction with Hip1.

For any given element of component of a described embodiment, any of the possible alternatives listed for that element or component may be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition. Each numerical parameter should be construed in light of the number of reported significant digits and by applying ordinary rounding techniques.

Any headings and subheadings used herein are for organizational purposes only and are not meant to be used to limit the scope of the description or the claims.

As used in this specification and the appended claims, the singular forms “a,” “an” and “the” do not exclude plural referents unless the context clearly dictates otherwise. Thus, for example, an embodiment referencing a singular referent (e.g., “an” inhibitor compound) may also include two or more such referents.

The embodiments disclosed herein should be understood as comprising/including disclosed components, and may therefore include additional components not specifically described. Optionally, the embodiments disclosed herein are free of one or more of the disclosed features. For example, any of the specific functional groups of the example inhibitor compounds and/or reagent compositions disclosed herein can optionally be omitted. Optionally, the embodiments disclosed herein are free of components that are not specifically described. That is, non-disclosed components may optionally be omitted from the disclosed embodiments. For example, functional groups and/or reagents that are not specifically described herein can optionally be omitted.

For purposes of disclosure, each of the originally filed claims recited herein shall be understood as disclosing any combination of claims as though each dependent claim was a multiple dependent claim of every preceding claim, except where clearly incompatible.

### EXAMPLES

### Example 1. Recombinant Expression and Purification of Hip1

An N-terminally truncated recombinant Hip1 was generated using PCR deletion mutagenesis using a full-length clone of Hip1 as template DNA. The truncated gene was subcloned into the glutathione S-transferase (GST) expression vector, pGEX-6P-1 (Amersham Biosciences). Overexpression of the Hip1-GST fusion protein was achieved in E. coli BL21 DE3 cells, cells lysed by sonication, and the fusion protein harvested as insoluble inclusion bodies. Using a modified method of Westling et al. the inclusion bodies were solubilized at 1 mg/mL in 8 M urea containing 0.05 M CAPS, 0.005 M EDTA and 0.18 M beta-mercaptoethanol with stirring at 25° C. for 45 minutes. To remove insoluble material, the mixture was centrifuged at 17,500×g for 30 min at 25° C. The clarified material was refolded by dialysis against 5.7× its volume of 0.05 M Tris-HCl, 0.005 M EDTA, 2 mM reduced glutathione, 0.4 mM oxidized glutathione, pH 7.3, at 4° C. with stirring. The dialysis buffer was replaced with fresh refolding buffer after 2 hours and protein was let refold for another 2 days. To concentrate the Hip1-GST fusion protein, it was centrifuged 20-30 min, 30,000×g, 4° C., and loaded onto 5×5 mL HiTrap Q FF columns equilibrated in 20 mM Tris-HCl, 10 mM NaCl, pH 8 (Buffer A). The column was washed at 1 mL/min for 70 min with Buffer A followed by a gradient elution of 10-100% Buffer B (20 mM Tris-HCl, 1.5 M NaCl, pH 8). To cleave the Hip1-GST fusion protein, 330 Units PreScission Protease® (Genscript) was added to 33 mg fusion protein in 20 mM Tris, 250 mM NaCl, pH 7.5, 1 mM DTT, 1 mM EDTA, 1% Tween 20 in a final volume of 4.8 mL. The reaction was rotated overnight at 4° C. N-terminal sequencing of recombinant Hip1 following PreScission Protease® cleavage indicates that the PreScission Protease® cleaves the fusion protein at the correct processing site yielding the expected N-terminus of Hip1: NH₂-GPLG. To separate Hip1 from GST, the PreScission Protease® reaction was dialyzed against 4 L of 20 mM Tris-HCl, 10 mM NaCl, pH 8 for 2-4 hours, 4° C. then exchanged with fresh dialysis buffer and let dialyze overnight. The dialysate was applied to 5×5 mL HiTrap Q FF columns equilibrated in 20 mM Tris-HCl, 150 mM NaCl, pH 8 (Buffer A), 4° C. The column was washed at 1 mL/min for 120 min with Buffer A followed by a linear gradient elution of 0-100% Buffer B (20 mM Tris-HCl, 1.5 M NaCl, pH 8) over 280 min. The Hip1 containing peak was concentrated to 13 mg/mL and applied to HiPrep 16/60 Sephacryl S-100 HR size exclusion column (GE Healthcare) to separate folded from misfolded and aggregated Hip1. Protein molecular weight and purity was assessed by SDS-PAGE.

### Example 2. Co-Crystallization of Hip1-Inhibitor Compound Complex and X-Ray Structure Solution

Hip1 was concentrated to approximately 10 mg/mL as determined by Bradford assay, using an Amicon Ultra-15 kDa MWCO spin concentrator (Millipore). To complex Hip1 with the inhibitor compound (NS-049-2), an approximate 1:1 molar ratio of inhibitor compound dissolved in 100% DMSO was added to concentrated Hip1 with a final DMSO concentration of 2%. The complex was incubated on ice overnight, then centrifuged at 14,000 rpm, 4° C. to remove precipitated protein. Crystal trials were set up using the hanging drop method with 4 μl drops, 2 mL mother liquor at 25° C. A fine screen of HR2-122 #15 (0.17 M ammonium sulfate, 0.085 M Na cacodylate trihydrate, pH 6.5, 25.5% w/v PEG 8,000, 15% v/v glycerol; Hampton Research) was conducted to determine optimal precipitant conditions. Large rods appeared in 5 days in buffer containing 25.5% PEG 8000, 0.12 M Ammonium Sulfate, 0.085 M Na Cacodylate trihydrate, pH 6.5, 15% v/v glycerol.

For crystallization of Hip1 with Inhibitor Compound 2, Hip1 was concentrated to approximately 10 mg/mL. To complex Hip1 with the inhibitor, an approximate 6:1 molar ratio of inhibitor dissolved in 100% DMSO was added to concentrated Hip1 with a final DMSO concentration of 2%. The complex was incubated on ice overnight, then centrifuged at 14,000 rpm, 4° C. to remove precipitated protein. Crystal trials were set up using the hanging drop method with 4 mL drops, 2 mL mother liquor at 25° C. A fine screen was conducted to determine optimal precipitant conditions. Large rods appeared in 12 days in buffer containing 23.5% PEG 8000, 0.22 M ammonium Sulfate, 0.085 M Na Cacodylate trihydrate, pH 6.5, 15% v/v glycerol.

Crystallographic data was collected to 2.7 Å on beamline 14-1 at the Stanford Synchrotron Radiation Lightsource (SSRL). Data were processed using HKL2000. The crystal belongs to space group P 3121 and contains one molecule in the asymmetric unit. The structure was solved by molecular replacement using apoHip1 as a search model (PDB ID 5UNO). Final refinement involved positional, individual b-factor, and TLS refinement utilizing secondary structure restraints and reference model restraints using apoHip1 (PDB ID 5UNO) as a reference model. Phaser-MR and Phenix.refine as implemented in the PHENIX software package was used for molecular replacement and refinement, respectively. Model building was performed using Coot. Structure validation was performed using Molprobity.

### Example 3. IC50 Values Against M. tuberculosis

Briefly, M. tuberculosis H37Rv expressing DsRed (Caroll et al., 2018) was grown in Middlebrook 7H9 medium containing 10% OADC (oleic acid, albumin, dextrose, catalase) supplement (Becton Dickinson) and 0.05% w/v Tween 80 (7H9-Tw-OADC) under aerobic conditions. Log phase bacteria were inoculated in assay plates containing compounds at a highest concentration of 200 μM using a 10-point two-fold serial dilution in 2% DMSO final concentration. Bacterial growth was measured by OD and RLU after 5 days of incubation at 37° C.

### Example 4. Intracellular M. tuberculosis Activity

M. tuberculosis H37Rv constitutively expressing DsRed (Caroll et al., 2018) was grown in Middlebrook 7H9 medium containing 10% v/v OADC (oleic acid, albumin, dextrose, catalase) supplement (Becton Dickinson) and 0.05% w/v Tween 80 (7H9-Tw-OADC) at 37° C. under aerobic conditions until log phase. Intracellular M. tuberculosis activity of inhibitor compounds was determined as described in (Manning et al., 2017). Briefly, RAW 264.7 cells were infected with log phase M. tuberculosis H37Rv constitutively expressing DsRed at a MOI of 1 for 24 hr at 37° C. in a humidified 5% CO₂ incubator. Infected cells were washed, harvested and inoculated in assay plates containing compounds and incubated for 3 days at 37° C. in a humidified 5% CO₂ incubator. Compounds were assayed at a highest concentration of 100 μM using a 10-point three-fold serial dilution in 1% DMSO final concentration. The cellular dye SYBR Green I (10,000×, Thermo Fisher) was added to assay plates at 5× final concentration and plates were imaged using an automated ImageXpress Micro XLS High Content Screening System (Molecular Devices) using FITC and Texas Red channels at 4× magnification. Raw data was normalized to negative control (1% DMSO) and expressed as % growth inhibition.

### Example 5. Activity and Low Toxicity in Cellular Assays

Cells were seeded in plates and incubated overnight in a humidified incubator at 37° C., 5% CO₂. Inhibitor compounds were added 24 hours post cell seeding to cells at a highest concentration of 100 μM using a 10-point three-fold serial dilution in 1% DMSO final concentration. After 72-hours of incubation, CellTiter-Glo® reagent (Promega) was added to plates and the relative luminescent units (RLU) were measured using a Synergy 4 plate reader (Biotek). Raw data were normalized using the average RLU value from negative control (1% DMSO) and expressed as % growth. Growth inhibition curves were fitted using the Levenberg-Marquardt algorithm. The IC₅₀ was defined as the compound concentration that produced 50% of the growth inhibitory response.

### Example 6. M. bovis as a Surrogate for Mtb

Since Mtb is a BSL-3 level pathogen, the investigators herein have identified M. bovis as a nonpathogenic surrogate strain to use for proof-of-concept studies. M. bovis contains a Hip1 orthologue with 100% amino acid identity and has been shown to be a suitable surrogate strain to use for the development of a rapid, POC, LFA diagnostic assay for the detection of Mtb through Hip1-mediated proteolysis of the novel molecular bait substrates, Dabcyl-Glu-Phe-Lys-Gln-Gly-Leu-Glu-(EDANS)-Arg and Cbz-Phe-Lys-Leu-pNa. The M. bovis reactions may reach the sensitivity required for a marketable diagnostic assay.

Next, studies were conducted to detect cleavage of a substrate similar to a molecular bait substrate, which are used in an LFA. Experiments determine if substrate cleavage is Hip1-dependent. FIG. 9 shows that M. bovis cleaves the FRET peptide substrate, UFNG1. Inclusion of inhibitors of cysteinyl proteases (E-64), aspartyl proteases (pepstatin), and metalloproteases (EDTA) indicated that members of these protease families do not inhibit cleavage of UFNG1 appreciably. Notably, the most significant inhibition was observed with the potent, Hip1-directed inhibitor compounds, Inhibitor Compound 1 and Inhibitor Compound 2, as well as the serine protease-directed inhibitor, AEBSF. The serine protease-directed inhibitor, PMSF, did not significantly inhibit cleavage of the substrate by Hip1. Furthermore, multiple studies have shown that PMSF is not an effective inhibitor of purified recombinant Hip1. Thus, PMSF may be an effective component to selectively inhibit contaminating endogenous serine proteases without significant inhibition of Hip1.

### Example 7. Sensitivity of the M. bovis Proof of Concept Assay

Using the conversion factor, OD₆₀₀ 1=3.13×10⁷ CFU/mL, approximately 450,000 M. bovis cells in the 10× cell culture dilution can be detected with UFGN2 in two hours (FIG. 16A) with a signal-to-noise ratio of ˜2, and 45,000 cells in the 100× cell culture dilution with a signal-to-noise ratio of ˜1.2. This is significant. Although the concentration of Mtb bacilli in the sputum of infected individuals ranges greatly across patients and depends on multiple factors, data from independent studies agree on average concentrations between 100,000 and 10,000,000 CFU/mL. Thus, the diagnostic assay herein may provide sufficient sensitivity for marketability. Notably, UFNG1 and UFNG2 have Kₘ values in the μM range (FIG. 10). The core (Inhibitor Compound 1: Cbz-Phe-Lys-Leu-CO—CO₂Me and Inhibitor Compound 2: Cbz-Phe-Lys-Glnlac-CO—CO₂Me) of the putative molecular bait for the development of a LFA have Ki values in the pM range. This is at least three orders of magnitude increase in binding potency compared to UFNG1 and UFNG2. Thus, the proof-of-concept studies that use UFNG1 and UFNG2 may significantly underestimate the sensitivity obtainable in an LFA, which utilizes derivatives of Inhibitor Compounds 1 and 2 as bait for Hip1. Moreover, if needed, the bait substrate sequence may be optimized for k_{cal}/Kₘ values via structure-based drug design and positional scanning combinatorial library analysis. Additionally, the poor signal to noise ratio may be mitigated by using colloidal gold antibodies rather than a fluorescent-base reporter system in the LFA prototype.

### Example 8. Chromogenic Substrate Versus Fluorescent Substrate

The novel, chromogenic substrate, Cbz-Phe-Lys-Leu-pNa provided herein is cleaved by M. bovis (FIGS. 11A and 11B). Chromogenic substrates are inherently less robust chemical reporters than fluorescence-based reporter systems. Current experiments, however, have achieved about the same level of detection of M. bovis cells with the chromogenic substrate as was achieved with the more robust fluorescent substrate, UFNG2 (FIGS. 16A and 16B). A positive test result was observed without the use of any instrumentation after incubating the chromogenic substrate for fifteen minutes with 5.3×10⁶ M. bovis cells (FIG. 11A). Notably, the chromogenic substrate has a much lower Kₘ=421+43 nM (FIGS. 12 and 13), which may explain its ability to detect roughly the same number of cells as UFNG2.

### Example 9. Cleavage of the Bait Substrate by Endogenous Human Proteases

Cleavage of the bait substrate by endogenous human proteases in sputum samples or saliva is a viable concern, since this would yield a false positive test. Hip1 is not encoded by the human genome; however, there are other endogenous proteases that could potentially generate a false positive test result. The main classes of proteases in the human lung are the serine, cysteinyl, aspartyl, and metalloproteases, all of which can function intra- or extracellularly. Additionally, there are proteases in human saliva. To prevent Hip-1 independent cleavage of the bait substrate by endogenous human proteases from the lung or saliva, sputum samples from patients are preincubated with a protease inhibitor cocktail. The cocktail includes EDTA (metalloprotease inhibitor), phenylmethylsulfonyl fluoride (PMSF; serine protease inhibitor), E64 (cysteine protease inhibitor), and pepstatin A (aspartyl protease inhibitor) to promote broad spectrum protection against endogenous human proteases or other contaminating proteases from microflora. Studies have shown that PMSF, a serine protease inhibitor, is a poor inhibitor of Hip1. Thus, PMSF may be added to the cocktail to selectively inhibit serine proteases without significant collateral inhibition of Hip1. Neither chymostatin nor leupeptin are included, since they both inhibit Hip1. Additionally, refinement of the bait substrate sequence through structure-based design may be used to increase the selectivity of the bait substrate for Hip1 over contaminating human proteases. Based the cocrystal structures herein of Hip1 bound with embodiments two potent inhibitor compounds herein, these investigators have generated the first atomic roadmap of the active site of Hip1, which can be used to refine the bait substrate for selectivity properties.

False negatives may arise if Mtb present in the lung does not express active Hip1. However, of 6,514 genomes obtained from clinical isolates around the world, 98.6% (6,420) encode for Hip1 and conserve the three key catalytic residues required for proteolytic activity. Though the presence of the gene does not guarantee activity, Hip1 has been shown to be important for infection. Therefore, false negatives are unlikely.

### Example 10: Diagnostic Assay Based on Inhibitor Compounds 1 and 2

This LFA is based on the potent, Hip1 recognition sequences from Inhibitor compounds 1 and 2 (Tables 1 and 2), which are modified to generate a “molecular bait” substrate for the detection of Mtb via Hip1-dependent cleavage. Hip1 is particularly suitable as a reporter for the presence of Mtb because it resides on the cell surface of the bacterium, thus has access to the molecular bait substrate for peptide bond hydrolysis (cleavage). Substituting the C-terminal, alpha keto methyl ester warhead with any natural or non-natural amino acid(s) or hapten (e.g. keyhole limpet hemocyanin, bovine serum albumin, or ovalbumin) can result in a peptide bond cleavable by Hip1.

We have demonstrated this with the development of several fluorescent substrates in which the alpha keto methyl ester warhead is replaced with the sequence Gly-Leu-Glu-(EDANS)-Arg (FIG. 5; UFNG2 and UFNG3). All FRET substrates that we have developed are cleaved by Mycobacterium bovis (M. bovis) cells, with UFNG2 being the most effective substrate in cell culture (FIG. 6). Additionally, the substrate sequence may be lengthened on the N-terminus using any natural or non-natural amino acid(s), protecting group, or hapten (e.g. keyhole limpet hemocyanin, bovine serum albumin, or ovalbumin).

Cleavage of the bait substrate (Xₙ₊₁-Phe-Lys-Glnlactam-Xₙ₋₁) sequence by Hip1 unmasks a hidden C-terminal epitope (Xₙ₊₁-Phe-Lys-Gln lactam-COO⁻). Analysis of the cocrystal structure of Hip1 bound with Inhibitor compound 2 reveals that there is ample room in the S4 pocket of the substrate binding site of Hip1 to lengthen the substrate, if required for epitope optimization (FIG. 7), since six amino acids are usually required to constitute a sufficient epitope. Conjugated to the test line antibody are colloidal gold nanoparticles or a similar detection system, which generate a visible (e.g., red) line on the test strip. At the test line, Xₙ₊₁-Phe-Lys-Gln lactam-COO⁻ was immobilized. At the control line, a capture antibody against the detection antibody was immobilized (FIGS. 8A and 8B).

If Mtb is present in the sputum sample, Hip1 cleaves the molecular bait substrate, Xₙ₊₁-Phe-Lys-Gln lactam-Xₙ₋₁, and liberates the analyte, Xₙ₊₁-Phe-Lys-Gln lactam-COO⁻, which is recognized and bound by the colloidal gold nanoparticle conjugated detection antibody. This prevents the binding of the detection antibody to the immobilized Xₙ₊₁-Phe-Lys-Gln lactam-COO⁻ at the test line, thus no red line appears (FIG. 8A, positive result). Bound detection antibody continues migration to the control line where an antibody against the detection antibody is immobilized, thus resulting in a red line, which indicates the assay is functioning properly.

If Mtb is absent from the patient sample, then there is no Hip1-dependent proteolytic cleavage of the bait substrate, Xₙ₊₁-Phe-Lys-Gln lactam-Xₙ₋₁, thus the hidden epitope is not unmasked. Consequently, the detection antibody remains unbound and can bind to the immobilized Xₙ₊₁-Phe-Lys-Gln lactam-COO⁻ at the test line resulting in a visible line (negative result, FIG. 8B). Unbound detection antibody continues migration to the control line where an antibody against the detection antibody is immobilized, thus resulting in a visible line, which indicates the assay is functioning properly.

### Results Summary

Since Mtb is a BSL-3 level pathogen, M. bovis was used as a nonpathogenic surrogate strain in proof-of-concept studies. M. bovis contains a Hip1 orthologue with 100% amino acid identity making it an ideal surrogate bacterial strain.

FIG. 9 shows that M. bovis cleaves the FRET peptide substrate, UFNG1. Inclusion of inhibitors of cysteinyl proteases (E-64), aspartyl proteases (pepstatin), and metalloproteases (EDTA) indicated that members of these protease families did not cleave UFNG1 appreciably. Notably, the most significant inhibition was observed with the potent, Hip1-directed inhibitors, Inhibitor compound 1 and Inhibitor compound 2, as well as the serine protease-directed inhibitor, AEBSF. Nonspecific (Hip1-independent) cleavage of UFNG1 by endogenous proteases in the presence of Inhibitor compounds 1, 2, and AEBSF is likely because the recognition sequence of UFNG1 has not been through a round of refinement for Hip1 selectivity optimization, as have the sequences of UFNG2 and UFNG3 (FIG. 5). The serine protease-directed inhibitor, PMSF, did not significantly inhibit cleavage of the substrate by Hip1. Furthermore, multiple studies have shown that PMSF is not an effective inhibitor of purified recombinant Hip1. Thus, PMSF can be used to selectively inhibit endogenous serine proteases without significant inhibition of Hip1.

A rough estimation of the sensitivity of the proof-of-concept assay has been established. Using the conversion factor, OD₆₀₀ 1=3.13×10⁷ CFU/mL approximately 450,000 M. bovis cells can be detected with UFNG2 in the 10× cell culture dilution in two hours (FIG. 16A) with a signal-to-noise ratio of ˜2, and 45,000 cells in the 100× cell culture dilution with a signal-to-noise ratio of ˜1.2. Although the concentration of Mtb bacilli in the sputum of infected individuals ranges greatly across patients and depends on multiple factors, data from independent studies suggests average concentrations between 100,000 and 10,000,000 CFU/mL. Thus, the assay provided herein enables the possibility a diagnostic assay with sufficient sensitivity for effective use in typical patients.

UFNG1 and UFNG2 have Kₘ values in the u M range (FIG. 10). Inhibitor Compound 1: Cbz-Phe-Lys-Gln-CO—CO₂Me and Inhibitor Compound 2: Cbz-Phe-Lys-Leu-CO—CO₂Me, adapted as molecular bait for the development of a LFA, have Ki values in the pM range. This represents at least three orders of magnitude increase in binding potency compared to UFNG1 and UFNG2. Thus, the proof-of-concept studies herein using UFNG1 and UFNG2 actually underestimate the sensitivity obtainable in an LFA, which utilizes derivatives of Inhibitor compounds 1 and 2 as bait for Hip1. Moreover, if needed, the bait substrate sequence may be optimized for k_{cal}/Kₘ values via structure-based drug design and positional scanning combinatorial library analysis. Additionally, the signal/noise may be improved by using colloidal gold antibodies rather than a fluorescent-base reporter system in the LFA prototype.

The novel, chromogenic substrate, Cbz-Phe-Lys-Leu-pNa (pNa=paranitroanaline) herein is demonstrated to be cleaved by M. bovis (FIGS. 11A and 11B). Chromogenic substrates are inherently less robust chemical reporters than fluorescence-based reporter systems. Interestingly, the results reported herein have achieved about the same level of detection of M. bovis cells with the chromogenic substrate as with the more robust fluorescent substrate, UFNG2 (FIGS. 16A and 16B). A positive test result can be seen without the use of any instrumentation after incubating the chromogenic substrate for fifteen minutes with 5.3×10⁶ M. bovis cells (FIG. 11A). Notably, the chromogenic substrate has a much lower Kₘ=421±43 nM (FIGS. 12 and 13), which may explain its ability to detect roughly the same number of cells as UFNG2.

M. bovis is a suitable surrogate strain to use for the development of a rapid, POC, LFA diagnostic assay for the detection of Mtb through Hip1-mediated proteolysis of the novel molecular bait substrates, Dabcyl-Glu-Phe-Lys-Gln-Gly-Leu-Glu-(EDANS)-Arg and Cbz-Phe-Lys-Leu-pNa. These assays exhibit sensitivity sufficient for a marketable and effective diagnostic assay, as demonstrated herein. Further provided herein is a novel, competitive LFA assay. Also disclosed herein is a novel protocol for selectively inhibiting contaminating, endogenous proteases. Thus, the assay presented herein may be optimized and translated into a rapid, point-of-care, LFA useful for the detection of Mycobacterium tuberculosis.

1. A compound for inhibiting a Hydrolase important for pathogenesis (Hip1) enzyme, the compound comprising the formula:
Prot-(X)ₙ—Z—(X)ₘ—Y
wherein
Prot is an optional N-terminal protecting group,
Z is a lysine or norarginine,
each X is independently any amino acid or amino acid derivative,
n is a number ranging from 1 to 10,
m is a number ranging from 1 to 10, and
Y is
(i) a reporter group, or
(ii) CO—CO₂R, wherein R is an alkyl, such that CO—CO₂R forms an alpha-keto alkyl ester.

2. The compound of claim 1, wherein Y is a reporter group, and wherein the reporter group comprises a chromophore or fluorophore.

3. The compound of claim 1, wherein Y is CO—CO₂R

4. The compound of claim 3, wherein R is a methyl or ethyl.

5. The compound of claim 1, wherein Prot comprises a pyrazine or a group that forms a carbamate with the N-terminal.

6. The compound of claim 1, wherein the N-terminal protecting group comprises a benzyloxycarbonyl (Cbz), tert-butyloxycarbonyl (Boc), or fluorenylmethyloxycarbonyl (Fmoc).

7. The compound of claim 1, wherein Xₙ comprises phenylalanine or tyrosine.

8. The compound of claim 1, wherein Xₘ comprise leucine, glutamine, a glutamine lactam, or norleucine.

9. The compound of claim 1, wherein the compound comprises Formula I or Formula II:

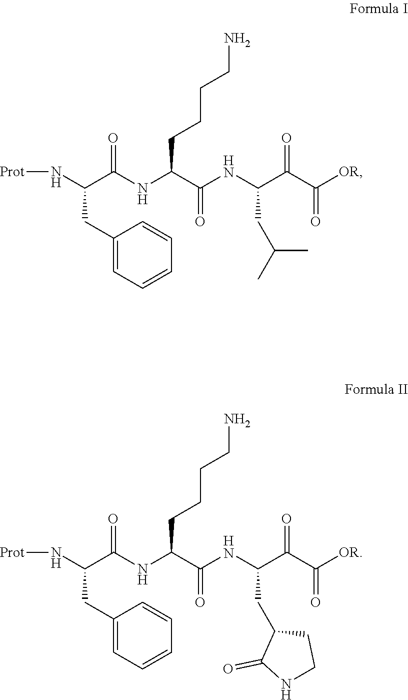

10. The compound of claim 1, wherein Xₘ does not include any glycine residues within two residues of Z in the C-terminal direction.

11. An assay kit for detecting the presence of Mycobacterium tuberculosis within a biological sample, the assay kit comprising:
the compound of claim 1, wherein Y is a reporter group; and
one or more reagents formulated for mixing with the biological sample.

12. The assay kit of claim 11, wherein the one or more reagents comprise one or more protease inhibitors.

13. The assay kit of claim 12, wherein the one or more protease inhibitors includes ethylenediaminetetraacetic acid (EDTA), phenylmethylsulfonyl fluoride (PMSF), E-64, and/or pepstatin A.

14. A Mycobacterium tuberculosis vaccine, comprising the compound of claim 1 mixed with the bacilli Calmette-Guerin (BCG) vaccine.

15. An enzyme complex comprising Hip1 bound to the compound of claim 1.

16. An assay kit for detecting the presence of Mycobacterium tuberculosis within a biological sample, the assay kit comprising:
(i) a sample pad configured to receive the biological sample and conduct the biological sample toward a test line;
(ii) a bait substrate comprising (X)ₙ—P₃—P₂—P₁—(X)ₘ, wherein
X is any amino acid, amino acid derivative, or hapten,
n is any whole number ranging from 1 to 10,
m is any whole number ranging from 1 to 10,
P₃ is phenylalanine or tyrosine,
P₂ is lysine or norarginine,
P₃ is leucine, glutamine, glutamine lactam, or norleucine,
wherein the bait substrate is configured to lose Xₘ in the presence of Hydrolase important for pathogenesis (Hip1) enzyme to form a cleaved epitope; and
(iii) a labelled detection antibody specific for the cleaved epitope,
wherein cleaved epitope formed from cleavage of the bait substrate competes for binding to the labelled detection antibody with cleaved epitope immobilized to the test line.

17. The assay kit of claim 16, wherein the labelled detection antibody comprises a gold nanoparticle label.

18. The assay kit of claim 16, wherein Xₙ and/or Xₘ comprises a hapten, and wherein the hapten comprises keyhole limpet hemocyanin, bovine serum albumin, and/or ovalbumin.

19. The assay kit of claim 16, further comprising a control line, wherein the control line includes an antibody that is specific to the labelled detection antibody.

20. A method of using the assay kit of claim 16 to test a biological sample for the presence of Mycobacterium tuberculosis, the method comprising:
contacting the biological sample to the sample pad; and
when the biological sample includes Hip1 enzyme, the Hip1 enzyme cleaving the bait substrate to form cleaved epitope that binds to the labelled detection antibody prior to the detection antibody reaching the test line, or
when the biological sample is free of Hip1 enzyme, the labelled detection antibody passing to the test line and binding to the cleaved epitope immobilized on the test line.
